(19) United States
(12) Patent Application Publication
(10) Pub. No.: US 2006/0270986 A1
(43) Pub. Date: Nov. 30, 2006

(54) Auto-injection device

(76) Inventors: Edgar Hommann, Grossaffoltern (CH); Benjamin Scherer, Uster (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/417,911

(22) Filed: May 4, 2006

Related U.S. Application Data
(63) Continuation of application No. PCT/CH04/00657, filed on Nov. 1, 2004.

(30) Foreign Application Priority Data
Nov. 5, 2003 (DE) 103 51 598.4

(51) Int. Cl. A61M 5/20

(52) U.S. Cl. 604/136

(57) ABSTRACT

A device for preparing an injection apparatus for making an injection, the device including a connecting element connectable to a protective cap of a vial or ampoule connectable to the injection apparatus, wherein the connecting element is operable to open the protective cap.

### CROSS-REFERENCE TO RELATED APPLICATION(S)

This application is a continuation of International Application No. PCT/CH2004/000657, filed on Nov. 1, 2004, which claims priority to German Application No. 103 51 598.4, filed on Nov. 5, 2003, the entire content of each of which is incorporated herein by reference.

### BACKGROUND

The present invention relates to devices for administering, delivering, injecting or dispensing substances, and to methods of making and using such devices. More particularly, it relates to such devices, including, for example, injection devices, injection pens, etc., for dispensing medicinal substances, such as insulin or hormones, in selected amounts or doses. More particularly, the present invention relates to an auto-injection device, which may be designed to be disposable and/or for a single use only.

FIG. 3 illustrates an injection device 10 with a needle 5 disposed in the injection device 10 and surrounded by a needle guard 1 and a needle guard cap 6. The needle guard 1 is biased in the forward or dispensing direction of the injection device 10, on the left-hand side of FIG. 3, by a needle guard spring 3 supported against the housing 14 of the injection device 10. To administer an injection, the needle guard cap 6 is removed. The injection device 10 is placed with the front end of the needle guard element 1 on the injection point and the needle guard element 1 must be pushed back against the force of the needle guard spring 3 by the indicated distance D in order to be able to insert the needle 5. The fact that the needle guard element 1 has to be pushed back against the force exerted by the needle guard spring 3 over the distance D, which is usually in the range of approximately 18 mm, means that a user has to push the injection device 10 onto the injection point, applying a relatively strong pressure, for quite a long time in order to administer the injection. If the ampoule 11 contains a substance or medicine which should not come in contact with metal—for example, the needle 5—for any length of time, it is necessary for the needle 5 to not be fitted on the ampoule 11 until shortly before the injection.

### SUMMARY

In one embodiment, the present invention comprises a priming device or tool for priming an injection device, such as an auto-injection device, which may be designed as a disposable part for one-off use, in order to administer an injection, wherein the injection device contains an ampoule with a substance to be dispensed, for example a medicine.

In some embodiments, the ampoule may be sealed and provided with a protective cap. Such protective caps typically consist of a rubber part which seals the ampoule, wherein the rubber part is secured on the ampoule by means of a plastic cap which may also contain a thread for a Luer needle. In some preferred embodiments, the plastic cap may be provided with a breaking point or area of weakness which may be broken, for example by turning or pulling, so the rubber part or seal can be removed, together with the broken-off plastic part, to open the ampoule. If a suitable connecting element for a needle is provided on the seal, then a Luer needle can be screwed or fitted on. In accordance with one embodiment of the present invention, the priming device or tool may be already connected to or can be connected to the seal or protective cap of the ampoule in the ampoule's initial state, such that the protective cap of the ampoule can be removed by turning and/or pulling the priming device or the tool, in order to enable a needle to be fitted or screwed on. The priming device in accordance with the present invention can be inserted into the injection device or is already provided in the injection device and, in some preferred embodiments, comprises a grip which is easy to grasp in the inserted state and can be turned by a user relative to the injection device to open the seal of the ampoule.

In some embodiments, the priming device can be located or is pushed in at the dispensing or front end of the injection device and projects out of the injection device, enabling a user to easily grasp and/or hold the priming device and turn it relative to the injection device, thus opening a seal of the ampoule, and enabling the user to take the seal out of the injection device, together with the priming device. In some embodiments, the priming device can be an approximately cylindrical, elongated element exhibiting an external diameter which is slightly smaller than the internal diameter of a needle guard element or a front end of the injection device.

Another embodiment of the present invention comprises a priming device or tool for priming an injection device, in particular an auto-injection device, wherein the priming device or tool has a holder element for a needle, e.g., a Luer needle, which can hold the needle for fitting it on or inserting it in the injection device and can release the needle once it has been fitted and/or screwed on, such that the priming device can then be removed from the injection device again, which has thus been primed for administering an injection by placing a needle onto an opened ampoule. Therefore, a priming device in accordance with the invention enables a needle to be fitted or screwed on in a region in the interior of an injection device that is protected by a housing or a needle guard element and difficult to access, and in which the needle can be connected to the injection device or to an ampoule, for example by a screw fitting, and the fixture or connection between the needle and the priming device can be released, for example by pulling the priming device off the needle connected to the ampoule or injection device. In some embodiments, the needle holder can be a simple receiving element, into which the needle is pushed, or can also comprise releasable retaining or connecting elements, such as for example catch lugs, clamping pieces or clips. The priming device can be designed such that a needle is already fitted in the priming device in the initial state or, alternatively, such that a needle can be inserted into the priming device and fitted onto the injection device together with the priming device.

In some preferred embodiments, the priming device is advantageously designed such that the needle, including a needle guard cap fitted on it, can be inserted or fitted into the priming device, wherein the priming device can hold the needle guard cap, for example by latching with the needle guard cap, once the needle has been inserted into the priming device, such that the needle can be fitted onto the injection device by means of the priming device and the priming device can be removed again from the injection device, together with the needle guard cap, thus exposing the needle in order to administer an injection.

In accordance with a preferred embodiment, the priming device comprises both the above-mentioned connecting element for connecting the priming device to an ampoule seal or protective cap, and the above-described holder element for a needle, wherein these two elements are provided at opposite ends of a generally rod-shaped priming device, such that the priming device can easily be turned by 180° once the ampoule has been opened to fit a needle onto the opened ampoule.

In some embodiments, the priming device comprises a mechanism for limiting a torque which is transmitted to the injection device by a user turning the priming device to screw on or attach a needle. The torque can be limited by designing the priming device in two parts, wherein one part of the priming device, which is gripped by a user, is connected or coupled to the other part by connecting elements, such as cams or other suitable structures, such that when a specific and pre-definable torque is exceeded, the connection between the two parts of the priming device is released or broken, enabling the two parts to rotate relative to one another. Thus, a torque acting on a first part of the priming device can only be transmitted to the second part to a specific maximum degree and a higher torque causes the first part to turn, or move or slide, further relative to the second part, which helps ensure that a needle is only attached to the injection device with a specific maximum force, and that too great a force or torque does not act on the injection device.

In some embodiments, the priming device may lock the injection device when it is inserted into the injection device, i.e., an injection procedure is precluded, and an injection can only be administered after the injection device has been fitted with an injection needle, wherein a locking ring (described below) can be provided which can only be removed from the injection device together with the priming device. In some preferred embodiments, the locking ring can only be removed after an injection needle has been fitted onto the injection device.

In another embodiment, the present invention comprises a locking ring for an injection device, which is disposed on a dispensing end of the injection device and/or is placed onto the injection device and retained by it, for example, by means of a latch mechanism. The locking ring may be designed to prevent a sliding movement of a triggering element for a triggering procedure, for example a movement relative to a housing or a needle guard, wherein the injection device is designed such that an injection procedure can only be initiated or administered after the sliding movement of the triggering element, in some embodiments, a triggering sleeve. To this end, the locking ring is fitted onto the front end of the injection device, for example onto the triggering sleeve and/or the needle guard, and connected to the injection device by a releasable connection formed by one or more catch elements. In some preferred embodiments, the connection between the locking ring and the injection device is releaseable by the priming device, after a needle has been fitted, wherein the connection can only be released after an injection needle has been securely connected to the injection device, for example by inserting the priming device into the injection device to a sufficient depth. To this end, cams or drivers can be provided on the external face of the priming device, which can release or unlock the lock or connection between the locking ring and the injection device when the priming device has been inserted to a sufficient depth, such that the locking ring can be removed from the injection device.

In some embodiments, the locking ring may be connected to the injection device such that the locking ring can rotate on the injection device but cannot slide axially until the connection between the locking ring and the injection device has been unlocked or released, for example by the priming device. Thus, it is possible for the locking ring to be coupled to the injection device by grooves in such a way that a rotation of the locking ring can be transmitted to the priming device, inserted into the injection device through the locking ring, such that a user can turn a priming device inserted into the injection device together with the locking ring to open the seal of an ampoule or to fit or screw on an injection needle.

In some preferred embodiments, the locking ring is mounted on the injection device in such a way that it cannot be moved in the axial direction without a connecting or bearing element being released, and such that an axial sliding movement of the locking ring, for example in order to remove the locking ring, is only possible once the corresponding connecting elements or retaining elements have been released, for example by the priming device described above.

In another embodiment, the present invention comprises an injection device with a locking ring as described above, wherein an injection cannot be triggered by operating a triggering button as long as the locking ring is situated on the injection device.

In another embodiment, the present invention comprises an injection device onto which a needle can be screwed or fitted or into which a needle can be inserted. In some embodiments, the injection device may already be connected to a needle and is provided with a needle guard element which is in a retracted position in an initial state and can be extended or pushed out, for example by a spring element, after an injection procedure, in order to be disposed around the needle to prevent any inadvertent contact with the needle. In some preferred embodiments, the needle guard element can only be extended from an initial retracted position to lock the needle, and can then be locked or latched in the extended or pushed-out position, for example by a catch connection or pawl which securely retains the extended needle guard such that it can no longer be retracted from the extended position to lock the needle.

Using such a driven needle guard provides that an injection device can be shorter than known injection devices with a needle guard which is retractable and extended in the initial state. Furthermore, the needle guard provided in accordance with the invention, which is not extended in the initial state, enables a shorter triggering path, requiring less force, for making an injection, because the needle guard does not first have to be retracted over a long distance against a force caused by a needle guard spring to release the needle. In accordance with the present invention, the needle is first extended out of the injection device, after which the needle guard element is pushed over the extended needle.

In some embodiments, the needle guard may be mounted on a syringe holder inside the injection device and is axially restricted in a forward position by means of a stop, wherein the needle guard is biased into the forward position by means of a spring element supported on the syringe holder.

In some embodiments, the present invention comprises a triggering sleeve coaxial with the needle guard, which projects beyond the needle guard and on which a locking ring as described above can be situated to prevent the triggering sleeve from any inadvertent sliding movement. Thus, the injection device is designed such that an injection triggering procedure can only be initiated after the triggering sleeve has been shifted relative to the needle guard, which can be achieved by pressing the injection device onto an injection point. Once shifted or inserted, for example to even with the front edge of a needle guard element, the triggering sleeve establishes a mechanical connection between a triggering button and a triggering mechanism, such that the triggering mechanism can only be operated after the triggering sleeve has been shifted by pressing the triggering button.

After the triggering button has been operated, an automatic triggering procedure is carried out, wherein an injection needle is extended or pushed forward out of the injection device by the force of a spring element to place it in a tissue. A displacement body, for example a plug, piston or the like, is then inserted into an ampoule, such that a substance contained in the ampoule is dispensed through the injection needle. After an injection procedure, the needle guard element is biased by a spring element towards the injection needle and automatically pushed over the injection needle when the injection device is removed from the injection point.

In some embodiments, the needle guard element may be provided with an indicator or display on its external face, for example a red, orange, yellow or another color, such that it is relatively easy to tell whether the injection device has already been used.

In some embodiments, a fixing element is provided, such as a catch, by which the needle guard can be latched or fixed in the extended position to secure or cover the injection needle to rule out as far as possible any inadvertent contact with the injection needle.

In one embodiment, the present invention comprises a needle insertion lock for a priming device or a tool as described above for priming an injection device, such as an auto-injection device. The lock helps ensure that the injection device can only be used after a needle has been placed on the injection device. The needle insertion lock can be inserted into or fitted onto the priming device and comprises at least one detecting element which can tell whether a needle or a needle guard cap fitted on a needle has been inserted into the priming device. In some preferred embodiments, the detecting element is comprises a blocking element, which protrudes radially outwardly from the priming device. The blocking element prevents the priming device from being inserted into the injection device if a needle or a needle guard cap fitted onto a needle has not been inserted into the priming device, wherein a needle inserted into the priming device or a needle guard cap containing a needle moves the blocking element to a position which enables the priming device to be inserted into the injection device.

In some embodiments, the detecting element comprises one or more elements which are biased inwardly, such as for example biased lugs, disposed in a circle around or abutting a needle guard cap and can be pushed or displaced radially outwardly from said needle guard cap. One or more spreading struts can be connected to the biased elements or biased lugs such that they can be shifted from a blocking position if an inserted needle guard cap is missing, into a releasing position if a needle guard cap is inserted.

In some embodiments, the needle insertion lock can be designed such that biased lugs are provided, biased radially inwardly, on an element which is designed annularly or on one or more stays of the needle insertion locking body which extend approximately parallel to the external face of the needle guard cap when a needle guard cap is inserted. Spreading elements can be connected to the biased lugs such that they are pressed outwardly about a pivot point of the needle insertion locking body when the biased lugs point radially inwardly due to their bias. When a needle guard cap or needle is inserted, the lugs are slightly pressed radially outwardly against the bias. The spreading elements coupled to the biased lugs are turned radially inwardly about the virtual pivot point formed by the needle insertion locking body, so they no longer protrude from the priming device and it is possible to insert the priming device into the injection device.

Since the needle insertion lock only enables the priming device to be inserted into the injection device when a needle or a needle guard cap fitted onto a needle has been inserted into the priming device, the locking ring described above can also only be removed from the injection device when a needle has been inserted into the injection device, such that the injection device can only be triggered after a needle has been correctly inserted into the injection device.

### BRIEF DESCRIPTION OF THE DRAWINGS

FIGS. 1A to 1F are cross-sections of an embodiment of an injection device in accordance with the present invention which is primed for administering an injection by a tool;

FIGS. 2A to 2F depict administering an injection with an injection device, and subsequently securing the injection needle;

FIG. 3 depicts an injection device in cross-section;

FIGS. 4A and 4B depict an embodiment of a priming device, with and without a needle inserted, in cross-sectional views;

FIG. 5 depicts the priming device illustrated in FIG. 4A, with a needle insertion lock inserted, in a perspective view; and

FIG. 6 depicts the priming device illustrated in FIG. 5, in an exploded view.

### DETAILED DESCRIPTION OF THE DRAWINGS

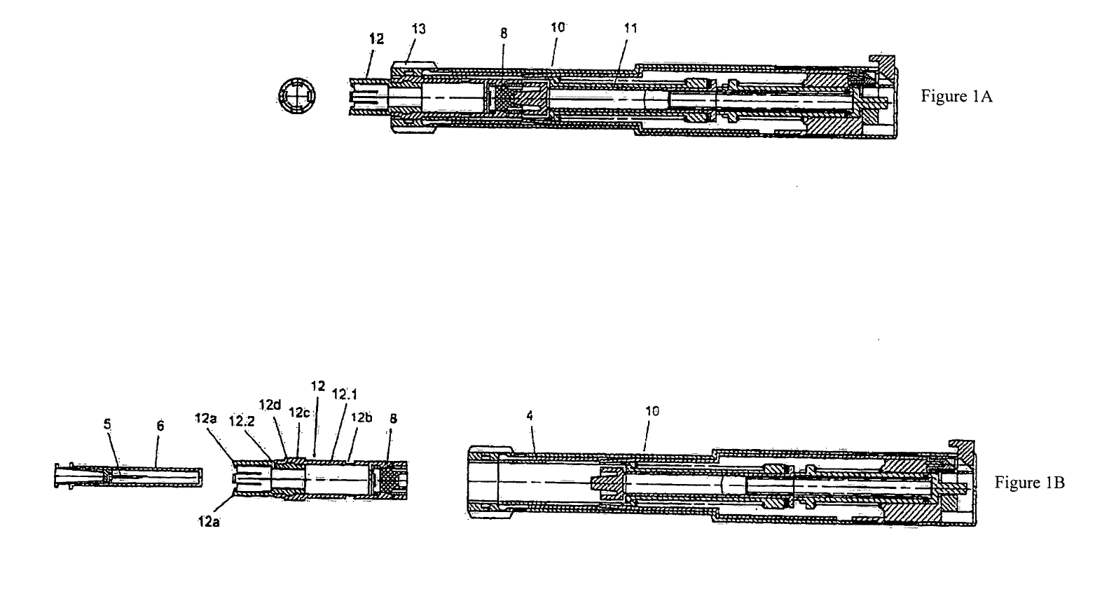

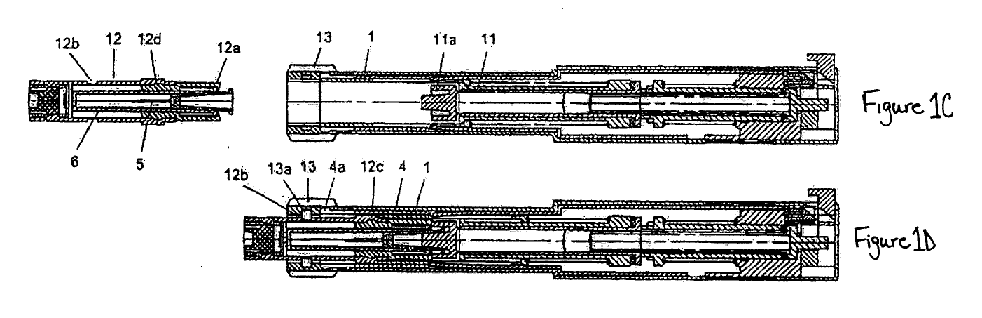

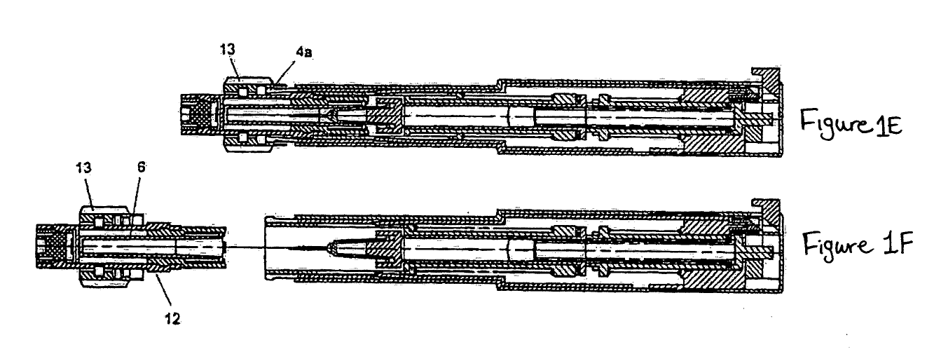

FIG. 1A illustrates a cross-section through an embodiment of the present invention, an auto-injection device 10, comprising an ampoule 11 which is sealed by a protective cap 8 consisting of a rubber part which seals the ampoule 11 and comprises a plastic cap which is connected to the ampoule 11 and simultaneously contains a thread for a Luer needle. The protective cap 8 is fixedly connected to the tool or priming device 12, which is mounted in the locking ring 13 such that it can be axially shifted and rotationally coupled. The tool 12 is connected via grooves or other suitable elements to the locking ring 13, which is axially connected or rotationally mounted on the injection device 10, such that a rotation of the locking ring 13 is transmitted onto the tool 12, which breaks a breaking point of the protective cap 8 and so opens the front end of the ampoule 11.

The tool 12, connected to the protective cap 8, can then be pulled out of the injection device 10 as illustrated in FIG. 1B, wherein the locking ring 13 remains axially connected to the injection device 10.

At the end of the tool 12 opposite the end comprising the protective cap 8, a Luer needle 5 together with a needle guard cap 6 fitted or carried on it can be inserted into the tool 12, wherein catch lugs 12a provided on the insertion opening of the tool 12 prevent the needle guard cap 6 from being able to be taken out of the tool 12 again, as illustrated in FIG. 1C.

If the tool 12 is turned by 180°, as illustrated in FIGS. 1C and 1D, the Luer needle 5 can be inserted into the injection device 10 through the locking ring 13 and screwed into an internal thread 11 a provided on the ampoule 11. This connects the Luer needle 5 fixedly to the front end of the ampoule opening, wherein the locking ring 13 is coupled to the tool 12, for example via grooves, such that a rotation of the locking ring 13 can be transmitted onto the tool 12 to screw on the Luer needle 5.

The tool 12 consists of two parts 12.1, 12.2 which are connected such that only a specific maximum torque can be transmitted from one part onto the other. The connection 12d between the two parts 12.1, 12.2, formed for example by catch lugs, is released when a pre-defined maximum torque is exceeded and causes one part to be able to rotate relative to the other, ensuring that the needle 5 is screwed on with only a defined torque below a pre-definable maximum torque. The turning can be accomplished by turning the locking ring 13 coupled to the tool 12, as illustrated in FIGS. 1D and 1E.

The tool 12 and locking ring 13 can then be removed from the injection device 10, the injection device 10 being primed for administering an injection with the ampoule 11 open and the needle 5 fitted, as illustrated in FIG. 1F.

After the protective cap 8 has been removed, the needle 5 is placed onto the ampoule 11, as illustrated in FIG. 1D, wherein a cam or projection 12c provided outside the tool 12 retracts the needle guard 1 provided inside the triggering sleeve 4 and so releases a cam 4a provided on the external face of the triggering sleeve 4. The sleeve 4 holds the locking ring 13 on the injection device 10, such that said cam 4a can be pushed radially inwardly and so releases the lock of the locking ring 13, such that the locking ring 13 can be removed from the triggering sleeve 4 and hence from the injection device 10. See FIGS. 1E and 1F.

The locking ring 13 can be removed by providing biased spring elements 13a in the locking ring 13, which push radially inwards and latch into corresponding recesses or cavities 12b on the external face of the tool 12, which fixedly connects the locking ring 13 to the tool 12 and is removed from the injection device 10 together with the tool 12 when the tool 12 is taken out, as illustrated in FIG. 1F. The cavities 12b are provided on the tool 12 such that the tool 12 has to be inserted into the injection device 10 at least far enough for the needle 5 to be securely fitted or fastened, before the spring elements 13a of the locking ring 13 are able to latch.

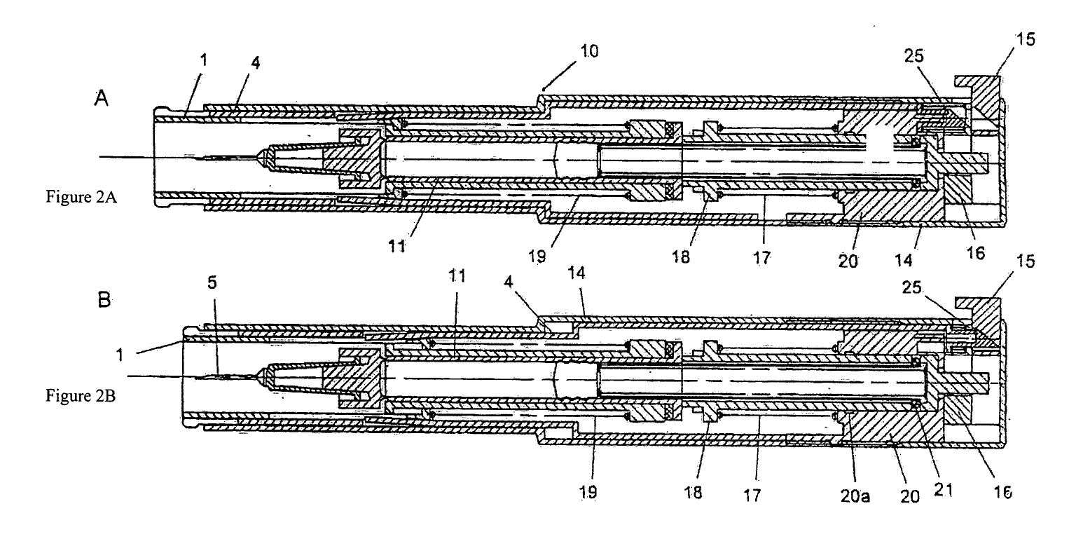

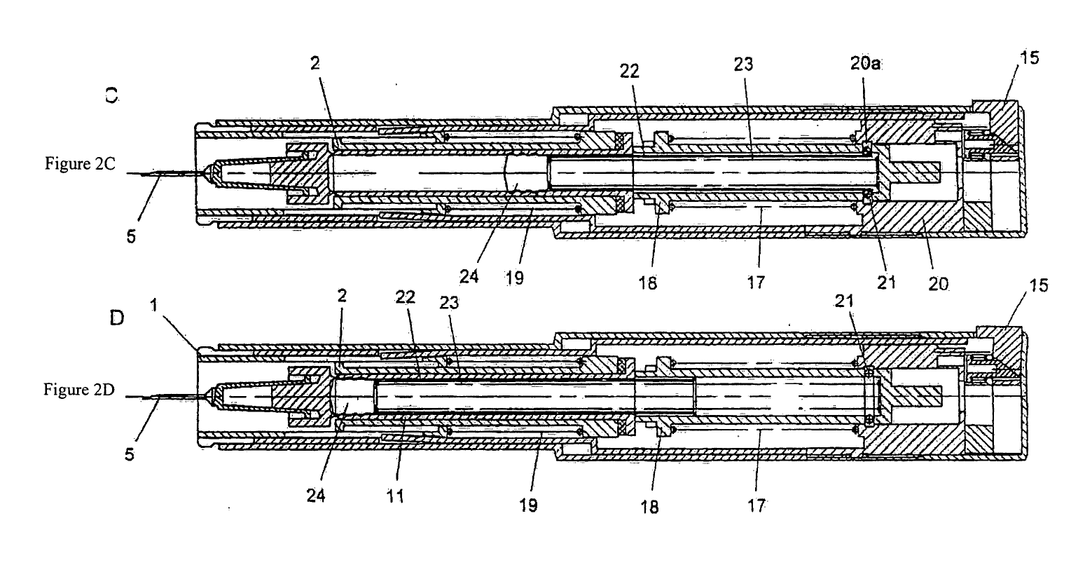

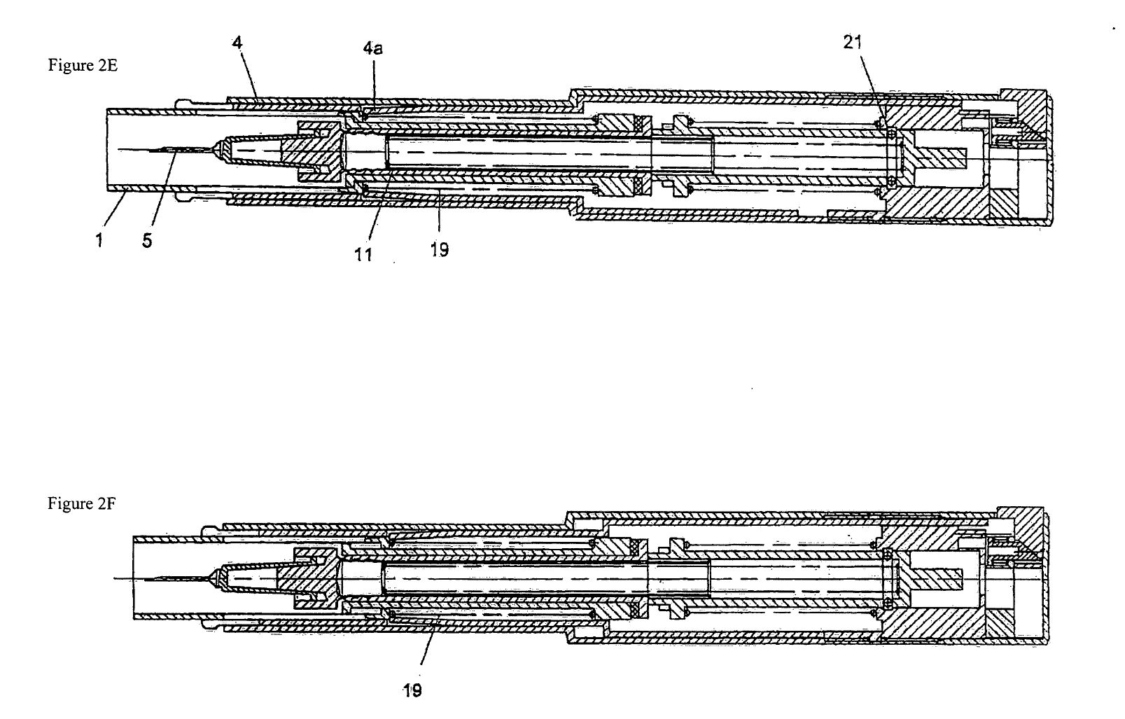

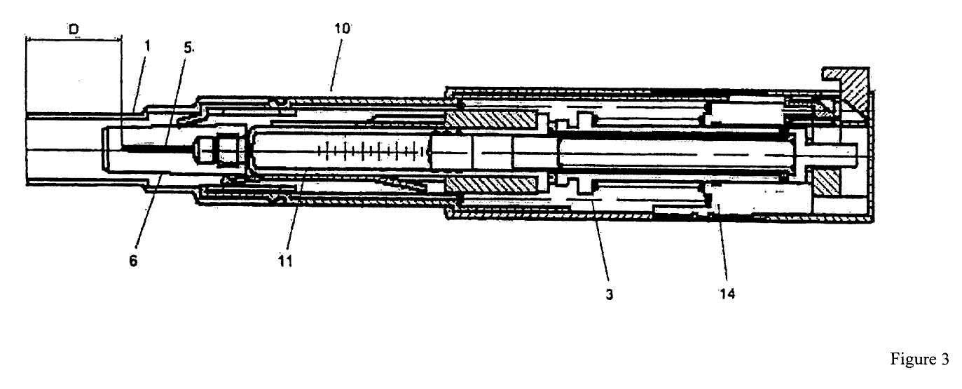

FIGS. 1F and 2A illustrate the injection device 10 primed for an injection procedure.

When the injection device illustrated in FIG. 2A is placed onto the injection point with a light pressure, the triggering sleeve 4 is retracted together with the needle guard 1, as illustrated in FIG. 2B.

An embodiment of an injection procedure will be described on the basis of FIGS. 2A to 2F. FIG. 2A illustrates the injection device 10 after the ampoule 11 has been opened and the needle 5 has been screwed on. In the initial state, or by being placed on an injection point, the triggering sleeve 4 is pushed back until the front end of the triggering sleeve 4 is approximately level or even with the front end of the needle guard 1, as illustrated in FIG. 2A. By being placed on an injection point, the triggering sleeve 4 is inserted into the ampoule housing 14 and, through the coupling element 25 shifting as illustrated in FIG. 2B, causes the triggering button 15—which until now it has been possible to press and release again without it having any effect on the injection device 10—to be coupled to the needle locking element 16 such that when the triggering button 15 is pressed in, the needle locking element 16 is pushed out of a position in which it secures the ampoule holder 18. The ampoule holder 18 is pushed by the needle ejecting spring 17, which is supported against the ampoule housing 14 or an ampoule holder guide 20, in the ejecting direction of the injection needle 5, which extends the needle 5—together with the ampoule 11 disposed behind the needle 5—out of the injection device 10, as illustrated in FIG. 2C. This simultaneously pushes the syringe holder 2 into a forward position, which tenses or compresses the needle guard spring 19 disposed between the syringe holder 2 and the needle guard 1.

When the ampoule holder 18 is pushed in the ampoule holder guide 20 far enough back that the latching elements 21 lie opposite the recesses or cavities 20a provided on the internal face of the ampoule holder guide 20, as illustrated in FIG. 2C, the latching elements 21 which are biased radially outwardly move into the cavities 20a of the ampoule holder guide 20, thus unlocking the plug holder or slide 22 and releasing it from the ampoule holder 18. See FIG. 2D. The injection spring 23 biased between the rear end of the ampoule holder 18 and the plug holder or slide 22 can therefore relax and push the plug 24 into the ampoule 11, as illustrated in FIG. 2D, which displaces the substance contained in the ampoule 11 and dispenses it through the extended needle 5.

When the injection device 10 is removed from the injection point after the injection has been administered, the needle guard 1—biased in the direction of the injection needle 5 by the biased needle guard spring 19—is pushed forwards out of the injection device 10 and over the needle 5, as illustrated in FIG. 2E, enabling the needle 5 to be secured or covered. An inwardly biased catch or pawl 4a, provided in the triggering sleeve 4, latches behind a rear stop of the needle guard 1 and thus locks it in the extended position.

In one embodiment, illustrated in FIG. 2F, the needle guard can be inserted slightly further when a pressure is applied, but without exposing the needle 5, until the triggering sleeve 4 coupled to the needle guard 1 is positioned on a projection of the ampoule holder guide 20.

If the needle guard 1 or the external face of the needle guard 1 exhibits a signal colour, for example red, the extended needle guard 1 can simultaneously also be used as an indicator, to indicate that the auto-injection device has already been used and cannot be used again.

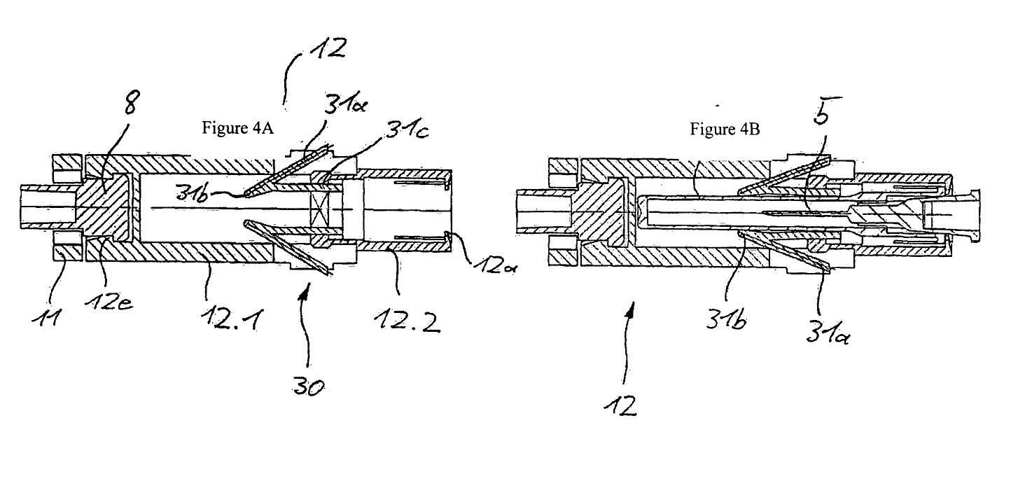

FIG. 4a illustrates a priming device or a tool 12 comprising a needle insertion lock 30 which can comprise all of the features described above, even if these are not illustrated in FIG. 4a. A protective cap 8 is held on a front face by catch lugs 12e, such that the protective cap 8 can be removed from the front face together with the tool 12. The needle insertion lock 30 is provided or inserted in the tool 12 such that the biased lugs 31b are biased radially inwardly and thus press the spreading elements or spreading struts 31a connected to them outwardly, wherein one face of the two abutting elements 31c of the needle insertion lock on which the biased lugs 31d and the spreading struts 31a are provided forms a pivot point of the lever formed by the biased lug 31d and the spreading strut 31a. The external ends or edges of the spreading struts 31a protrude laterally beyond the tool 12, as illustrated in FIG. 4A, and so prevent the tool 12 from being inserted into the injection device 10, since the protruding ends of the spreading struts 31a abut against the front face of the injection device 10 or the front face of the locking ring 13.

If a Luer needle 5 is inserted into the tool 12, on which a removable needle guard cap 6 is fitted, then the biased lugs 31b biased inwardly are pushed outwardly by the needle guard cap 6, as illustrated in FIG. 4B. A force directed radially inwardly acts on the spreading struts 31a connected to the biased lugs 31b, which pushes the spreading struts 31a into the tool 12 such that they no longer protrude out of the tool 12. Thus, the tool 12 can then only be inserted into the injection device 10 when a needle 5 has been inserted into the tool 12.

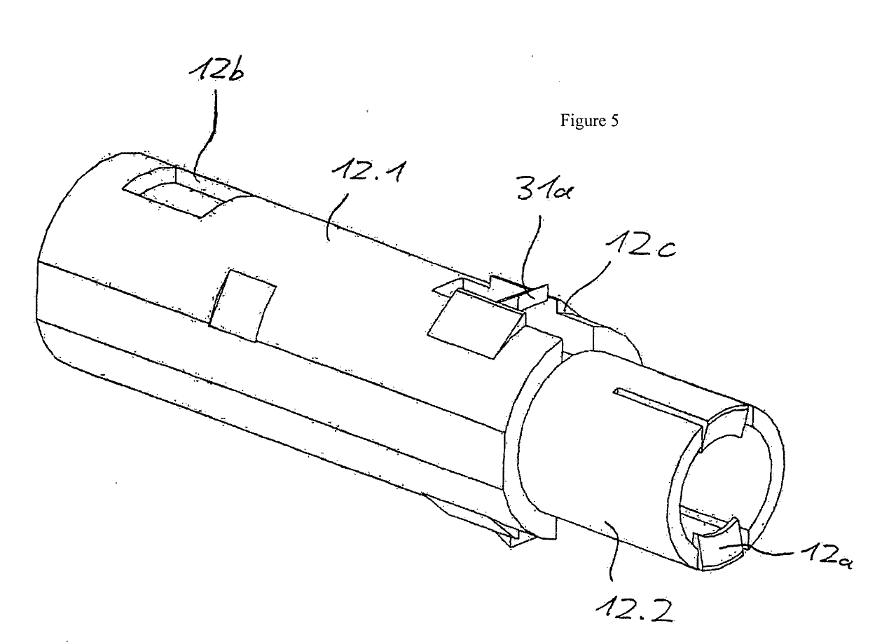

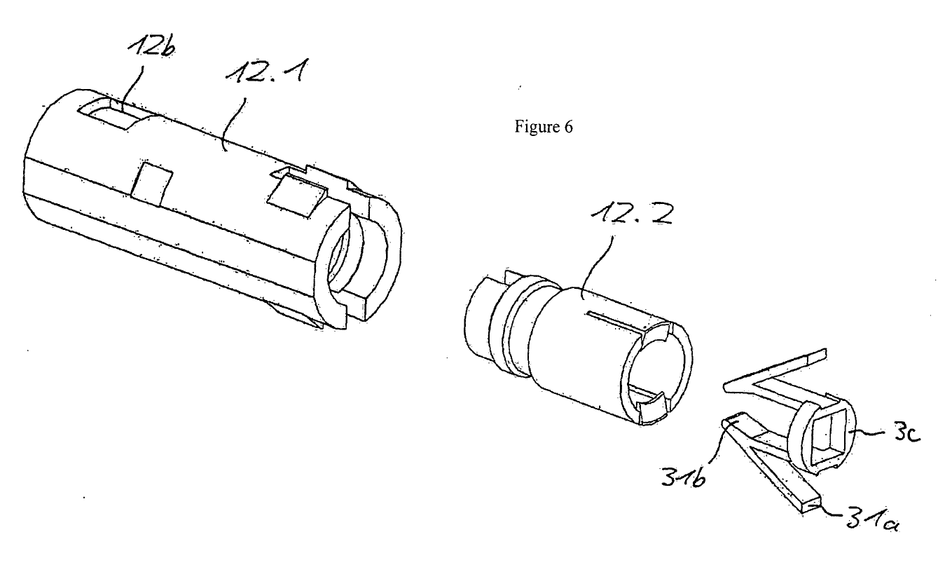

FIGS. 5 and 6 illustrate the tool 12 illustrated in cross-section in FIG. 4A, but in a perspective and exploded view, respectively. The tool 12 comprises the recesses or cavities 12b described above, into which biased spring elements 13a of the locking ring 13 can latch to fixedly connect the locking ring 13 to the tool 12 and to enable the removal of the locking ring 13 together with the tool 12 when the tool 12 is taken out of the injection device 10.

The external faces of the spreading struts 31a can protrude beyond the cams 12c illustrated, which can also be offset with respect to the external faces of the spreading struts 31a and, as described above, can retract the needle guard 1 provided inside the triggering sleeve 4 when the tool 12 is inserted into the injection device 10.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A priming device for priming an injection device for an injection, comprising a connecting element operably connectable to one of a seal or protective cap of an ampoule to open the ampoule.

2. The priming device according to claim 1, wherein the priming device is carried at a dispensing end of the injection device.

3. A priming device for priming an injection device for an injection, comprising a retaining element for a needle.

4. The priming device according to claim 3, further comprising a fixing element for retaining a needle guard cap in or on the priming device.

5. The priming device according to claim 1, further comprising a retaining element for retaining at least one of a needle or a needle guard on the priming device.

6. The priming device according to claim 5, wherein the connecting element and the retaining element are at opposite ends of the priming device.

7. The priming device according to claim 1, further comprising a torque limiting device.

8. The priming device according to claim 3, further comprising a torque limiting device.

9. An injection device comprising a locking ring releasably connected to the injection device for preventing a triggering of the injection device from being released.

10. The injection device according to claim 9, wherein the locking ring is rotatable relative to the injection device.

11. The injection device according to claim 10, wherein the locking ring is removeable from the injection device by a priming device.

12. The priming device according to claim 1, further comprising a needle insertion lock comprising at least one needle insertion detecting element connected to a blocking element which prevents the priming device from being coupled to the injection device if a needle or a needle guard element is not coupled to the priming device.

13. The priming device according to claim 12, wherein the needle detecting element comprises a biased lug connected to a spreading strut and shiftable by an inserted needle or needle guard cap whereby the spreading strut is shifted into a releasing position.

14. The priming device according to claim 13, wherein the sifting of the spreading strut is generally radially into the priming device.

15. The needle insertion lock according to claim 13, wherein the biased lug is biased radially inwardly and is connected to the needle insertion lock such that the biased lug is moveable about a virtual pivot point formed by the needle insertion lock, such that the spreading strut connected is moved in a direction radially opposite to the movement direction of the biased lug.
